(19) United States
(12) Patent Application Publication
(10) Pub. No.: US 2009/0092593 A1
(43) Pub. Date: Apr. 9, 2009

(54) THERAPEUTIC DRUG FOR HEART DISEASE AND VIRUS DISEASE

(75) Inventor: Akira Matsumori, Osaka (JP)

Correspondence Address: OBLON, SPIVAK, MCCLELLAND MAIER & NEUSTADT, P.C., 1940 DUKE STREET, ALEXANDRIA, VA, 22314, US

(73) Assignees: Akira Matsumori, Minoh-shi (JP); YAMASA CORPORATION, Choshi-shi (JP)

(21) Appl. No.: 12/064,543

(22) PCT Filed: Aug. 22, 2006
(86) PCT No.: PCT/JP2006/316419

§ 371 Date: Feb. 22, 2008

(30) Foreign Application Priority Data
Aug. 23, 2005 (JP) 2005-240803

(51) Int. Cl. A61K 39/395

(52) U.S. Cl. 424/130.1

(57) ABSTRACT

The present invention is related to provide a therapeutic drug for heart diseases and viral diseases. The invention provides a therapeutic drug for heart diseases and viral diseases, comprising a free immunoglobulin light chain or a constitutive polypeptide thereof as an active ingredient.

### TECHNICAL FIELD

The present invention relates to a therapeutic drug for heart diseases such as heart failure, cardiomyopathy, myocarditis, and fulminant myocarditis and for viral diseases.

### BACKGROUND ART

Heart failure is a pathological condition in which the heart fails to feed a sufficient amount of blood for maintaining the metabolism of tissues because of a disorder in its pumping action, and is caused by an ischemic heart disease such as myocardial infarction or angina pectoris, hypertension, cardiomyopathy, myocarditis, or a similar disease. Cardiomyopathy is a disease involving a disorder in cardiac muscle which provides the heart with a pumping action, and is classified into hypertrophic cardiomyopathy (thickening of cardiac muscle), dilated cardiomyopathy (thinning of cardiac muscle), restrictive cardiomyopathy (inhibition of dilatation), etc. Myocarditis is a disease involving inflammation of cardiac muscle, which is believed to be often caused by a virus.

At present, the means for treating heart failure, cardiomyopathy, myocarditis, and fulminant myocarditis is selected in accordance with the symptoms of these diseases. Examples of the therapeutic means include drug therapies employing a drug such as a heart stimulant, a diuretic agent, catecholamine, a vasodilator, an angiotensin-converting enzyme inhibitor, an angiotensin receptor antagonist, a β-blocker, an anti-arrhythmic agent, or an anti-inflammatory agent; and implantation of a cardiac pacemaker. In addition to these conventional therapeutic means, there is also demand for a therapeutic method based on a new strategy.

Meanwhile, a free immunoglobulin light chain (free light chain: FLC) which is dissociated from an immunoglobulin heavy chain is known to be excessively produced in pathologic states, particularly in myeloma. Such an FCL level increase is also reported in immunological diseases such as sarcoidosis (Non-Patent Document 1), Sjoegren's syndrome (Non-Patent Document 2), systemic lupus erythematosus (Non-Patent Document 3), multiple sclerosis (Non-Patent Documents 4 and 5), viral or bacterial meningitis (Non-Patent Document 4), AIDS (Non-Patent Documents 4 and 6), corneal grafting (Non-Patent Document 7), and rheumatoid arthritis (Non-Patent Documents 8 and 9).

Previous studies also revealed that the blood FLC level increases in such diseases as multiple sclerosis and rheumatoid arthritis, which are thought to be caused by mast cells (Non-Patent Documents 8 and 10). Another study revealed that the free kappa light chain level in blood is higher in atopic and non-atopic adult bronchitic asthma patients as compared with healthy subjects (Non-Patent Document 11). Peptide F991, which is formed of nine amino acids and serves as an FLC antagonist, is reported to be effectively suppressing respiratory obstruction and pneumonitis in non-atopic asthma model mice (Non-Patent Document 11). Also, FLC plays an important role in the onset of mast-cell-dependent contact allergy, and F991 (an FLC antagonist) can prevent the onset thereof (Non-Patent Document 12). However, there have never been elucidated effects of FLC on viral diseases, heart failure, cardiomyopathy, myocarditis, fulminant myocarditis, and other diseases.

Non-Patent Document 1: Romer F. K. et al., Eur. J. Respir. Dis. 1984; 65: 292-295 Non-Patent Document 2: Moutsopoulos H. M. et al., J. Immunol. 1983; 130: 2663-2665 Non-Patent Document 3: Hirohata et al., J. Rheumatol. 1986; 13: 715-721 Non-Patent Document 4: Fagnart O. C. et al., J. Neuroimmunol. 1988; 19: 119-132 Non-Patent Document 5: Lamers K. J. et al., J. Neuroimmunol. 1995; 19: 119-132 Non-Patent Document 6: Contini C. et al., J. Neuroimmunol. 2000; 108: 221-226 Non-Patent Document 7: Solling K. et al., Scan. J. Clin. Lab. Invest. 1978; 38: 369-373 Non-Patent Document 8: Solling K. et al., Acta Med. Scand. 1981; 209: 473-477 Non-Patent Document 9: Cooper A. et al., Ann. Rheum. Dis. 1968; 27: 537-543 Non-Patent Document 10: Lee D. M. et al., Science 2002; 297: 1689-1692 Non-Patent Document 11: Kraneveld A. D. et al., Proc. Natl. Acad. Sci. USA. 2005; 102: 1578-1583 Non-Patent Document 12: Redegeld F. A. et al., Nat. Med. 2002; 8: 694-701

### DISCLOSURE OF THE INVENTION

### Problems to be Solved by the Invention

An object of the present invention is to provide a new therapeutic drug for heart diseases such as heart failure, cardiomyopathy, myocarditis, and fulminant myocarditis and for viral diseases.

### Means for Solving the Problems

In the aforementioned bronchitic asthma, the free kappa light chain level increases, but the free lambda light chain does not increase. Since an FLC antagonist is effective for bronchitic asthma, FLC is conceived to aggravate the disease. Meanwhile, the present inventor previously found that the free kappa light chain level and free lambda light chain level in blood increase in patients of a viral disease, heart failure, cardiomyopathy, or myocarditis, and that the lambda chain level increases more as compared with the kappa chain level and the kappa/lambda ratio decreases in these patients (Japanese Patent Application No. 2005-114350). The further study of the inventor has revealed that the cardiac muscle lesion can be significantly mitigated and the survival rate can be significantly improve by administration of a free immunoglobulin light chain in encephalomyocarditis viral myocarditis (heart failure) model mice. Thereby the inventor has found that the free immunoglobulin light chain is useful as a therapeutic drug for heart diseases. The inventor has also revealed that the HCV antigen level of HCV-infected cells can be significantly reduced through administration of a free immunoglobulin light chain to the cells, and thereby found that the free immunoglobulin light chain is useful as a therapeutic drug for viral diseases.

Accordingly, the present invention provides a therapeutic drug for a heart disease and for a viral disease comprising, as an active ingredient, a free immunoglobulin light chain or a constitutive polypeptide thereof.

### Effects of the Invention

According to the therapeutic drug of the present invention, lesions and survival rate of patients of severe heart diseases such as heart failure, cardiomyopathy, myocarditis, and fulminant myocarditis can be improved, and the virus antigen level of patients of various viral diseases such as viral hepatitis can be reduced, to thereby treat the viral diseases. Thus, the invention can provide a new therapeutic means for the treatment of these diseases. The present invention can also realize the treatment of myocarditis, pericarditis, and other viral diseases.

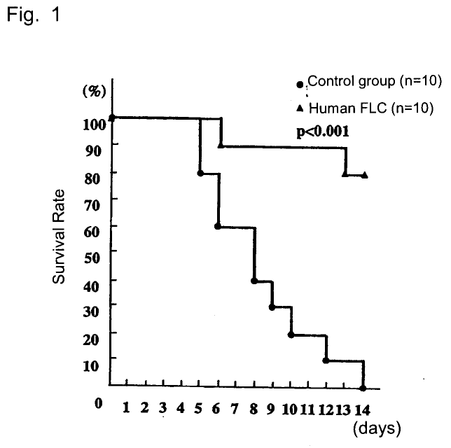

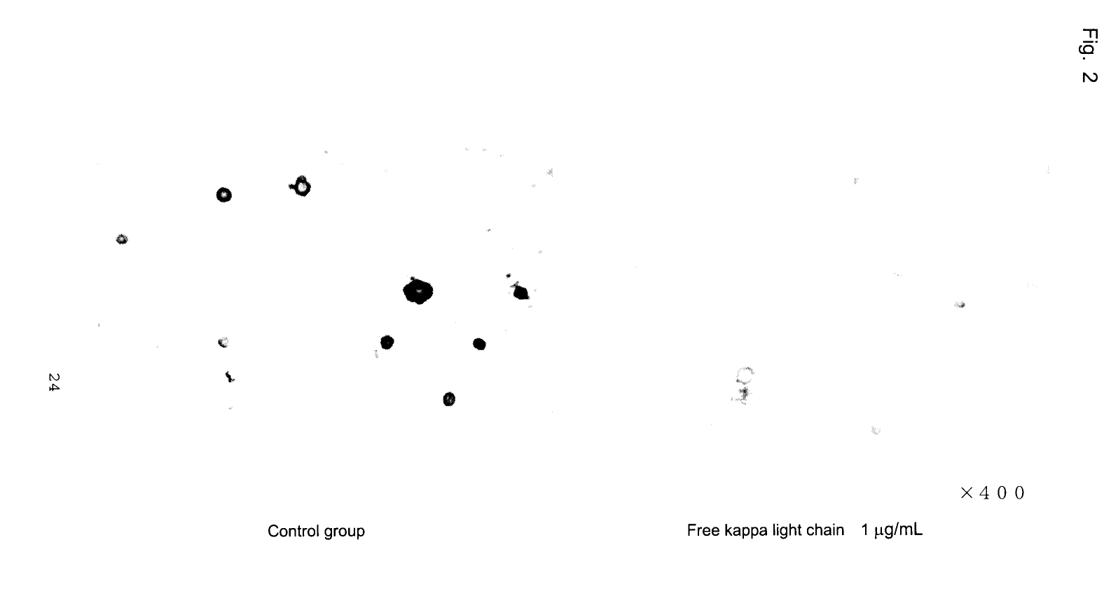

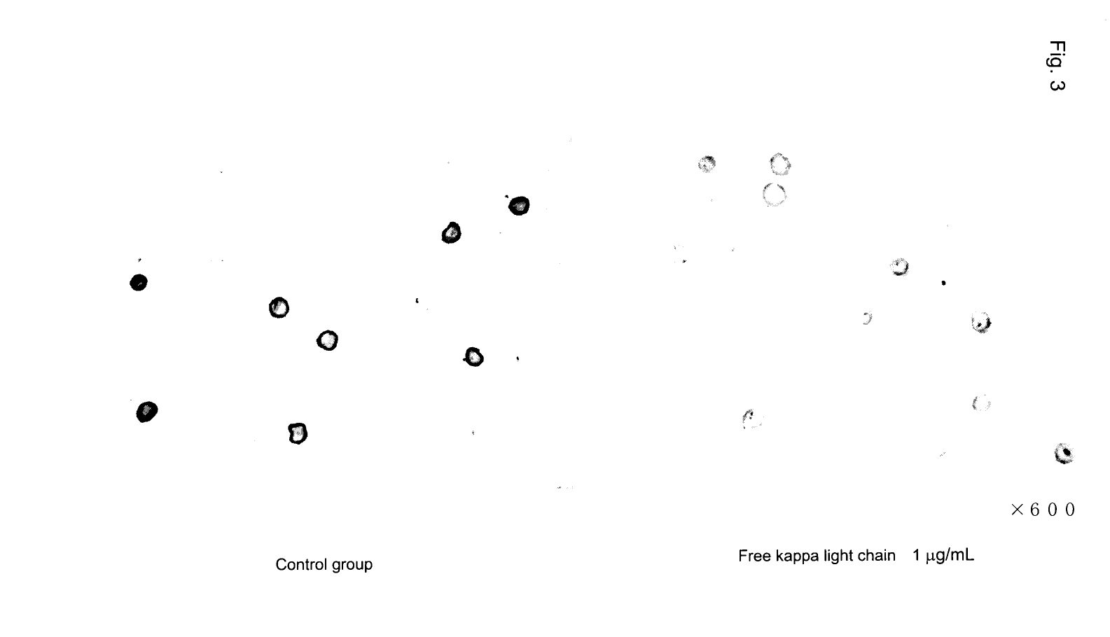

### BRIEF DESCRIPTION OF THE DRAWINGS

[FIG. 1] A graph showing changes in survival rate of encephalomyocarditis viral myocarditis (heart failure) models through administration of a free kappa light chain.

[FIG. 2] Photographs showing an antiviral effect of a free immunoglobulin kappa light chain on peripheral blood mononuclear leucocytes of patients infected with hepatitis C virus.

[FIG. 3] Photographs showing an antiviral effect of a free immunoglobulin kappa light chain on THP-1 cells infected with hepatitis C virus.

### BEST MODES FOR CARRYING OUT THE INVENTION

The free immunoglobulin light chains (FLCs) employed in the therapeutic drug of the present invention include a kappa chain and a lambda chain. Of these, the kappa chain is preferably employed. A mixture of a kappa chain and a lambda chain may also be employed. When such a mixture is employed, a mixture having a high kappa/lambda ratio; i.e., a high kappa chain content, is preferably used. Preferably, the kappa/lambda ratio is 1 or higher, particularly preferably 2 or higher.

The FLC constitutive polypeptide is preferably a polypeptide constituting a kappa chain. When a mixture of a kappa chain constitutive polypeptide and a lambda chain constitutive polypeptide is employed, a mixture having a high kappa chain constitutive polypeptide content is preferably employed. In this case, the kappa/lambda ratio is also preferably 1 or higher, particularly 2 or higher.

These FLCs or constitutive polypeptides thereof may be obtained through, for example, separating them from urine or blood of human subjects, gene recombination, or collecting them from myeloma cells. FLCs may be separated from urine or blood samples by means of, for example, electrophoresis or gel filtration (Solomon A., Methods Enzymol. 1985; 116:101-121).

As shown in the Referential Example and the Examples hereinbelow, in heart failure, cardiomyopathy, myocarditis, fulminant myocarditis, and viral diseases, the free kappa light chain blood level and the free lambda light chain level in blood increase, and the kappa/lambda ratio decreases. When a free kappa light chain is administered to viral myocarditis (and heart failure) models, survival rate and the cardiac muscle lesion of the models can be significantly improved. Therefore, an FLC, particularly a kappa chain, is a useful therapeutic drug for heart diseases such as heart failure, cardiomyopathy, myocarditis, and fulminant myocarditis, and for viral diseases. When a free kappa light chain is administered to virus-infected cells, the virus antigen level of the cells is reduced. Therefore, an FLC, particularly a kappa chain, is a useful therapeutic drug for various viral diseases. Herein, these viral diseases include diseases caused by a pathogenic virus belonging to DNA viruses or RNA viruses. Examples of such pathogenic viruses include the following:

DNA viruses: Poxvirus, Herpesvirus, Adenovirus, and Parvovirus; and

RNA viruses: Reovirus, Togavirus, Coronavirus, Rhabdovirus, Paramyxovirus, Orthomyxovirus, Bunyavirus, Arenavirus, Retrovirus, Picornavirus, and Calicivirus.

In particular, the therapeutic drug of the present invention is preferably employed for the treatment of a patient suffering from viral myocarditis caused by an RNA virus or a hepatitis virus, or suffering from a viral disease induced by the viral myocarditis, and prevention of such diseases. Examples of particularly preferred target RNA viruses include Orthomyxovirus and Picornavirus.

Specific examples of viral diseases induced by viral myocarditis include viral hepatitis (type A, type B, type C, type E, type G, and type TTV), adenovirus infections, influenza, viral pneumonia, viral bronchitis, herpes infections (herpes simplex, EB virus (infectious mononucleosis), and herpes zoster), polio, AIDS (HIV infections), adult T cell leukemia (ATL), papilloma, measles, rubella, exanthema subitum, infectious erytheme, viral encephalitis, viral meningitis, cytomegalovirus infections, mumpus, chicken pox, lyssa, viral enteritis, viral pericarditis, Coxsackievirus infections, echovirus infections, hemorrhagic fever with renal syndrome, and Lassa fever. Among these viral diseases, the therapeutic drug of the present invention is preferably applied to viral hepatitis (type A, type B, type C, type E, type G, and type TTV), adenovirus infections, influenza, herpes infections, viral encephalitis, cytomegalovirus infections, viral enteritis, and viral pericarditis. The therapeutic drug of the present invention is also effective for the treatment of these heart diseases and viral diseases showing a reduced FLC kappa/lambda ratio.

The therapeutic drug of the present invention may be administered, for example, intravenously, subcutaneously, intramuscularly, or intraperitoneally. Examples of drug compositions suitable for these administration routes include injection solution, lyophilized drug products, and powders. For producing these compositions, an FLC or a constitutive polypeptide thereof may be mixed with a solvent, a solution adjuvant, a stabilizer, etc.

The dose of the therapeutic drug of the present invention, which varies depending on the type, symptoms, etc. of the disease, is preferably 0.1 mg to 10 g/day/adult, as reduced to free kappa light chain.

### EXAMPLES

The present invention will next be described in more detail by way of Referential Example and Examples, which should not be construed as limiting the invention thereto.

### Referential Example 1

### (1) Serum Specimens

Serum specimens tested in this experiment were collected from healthy volunteers, heart failure patients, cardiomyopathy patients, and myocarditis patients.

### (2) FLC-Specific Monoclonal Antibody

An FLC-specific monoclonal antibody was produced through immunizing BALB/c mice with FLC (product of Bethyl laboratories, Montgomery, Tex.) (J. Immunol. Methods, 275, 9-17. (2003)). An F(ab′)₂ fragment of the produced antibody was labeled with horseradish peroxidase (HRP) (J. Histochem. Cytochem., 22, 1084-91. (1974)).

### (3) FLC Assay

FLC assay was performed according to literature procedures (J. Immunol. Methods, 2003, 275, 9-17). Specifically, a specimen or a standard concentration solution (each 100 μL) was added to a 96-well microplate (Nunc) coated with the FLC-specific monoclonal antibody, followed by reaction at room temperature for two hours. After washing, an HRP labeled anti-kappa light chain antibody and an HRP-labeled anti-lambda light chain antibody (each 100 μL) serving as secondary antibodies were added to the microplate, followed by reaction at 37° C. for 30 minutes. After reaction, the microplate was washed with PBS containing 0.05% Tween 20, and the reactions were allowed to develop color by dispensing to each well a color developing solution (OPD, product of Sigma) (each 100 μL). The color development was terminated with 1N sulfuric acid, and absorbance at 492 nm was measured. In the assay, 50 mM Tris-HCl (pH 7.5) containing 1% bovine serum albumin was employed as a buffer.

### (4) Assay Results

(4-1) Table 1 shows the results of FLC assay for heart failure patients (63 cases) (from December 2002 to November 2004).

TABLE 1

| | n | kappa (mg/L) | lambda (mg/L) | kappa/lambda |
|---|---|---|---|---|
| Heart failure | 63 | 40.8 ± 39.2* | 93.2 ± 67.9* | 0.38 ± 0.22* |
| Healthy subjects | 17 | 27.9 ± 5.3 | 43.6 ± 8.8 | 0.66 ± 0.16 |

Mean ± standard deviation,
*p < 0.0001

(4-2) Of all the patients, 17 patients who had undergo measurement of NT-proBNP level and SCF level were investigated in terms of correlation with FLC levels. The results are shown in Table 2.

TABLE 2

| | NT-proBNP(y) | | | SCF(y) | | |
|---|---|---|---|---|---|---|
| X | | r² | P | | r² | p |
| Kappa | y = 280x − 4920 | 0.511 | 0.0013 | y = 12.9x + 381 | 0.3518 | 0.012 |
| Lambda | y = 51.6x − 455 | 0.806 | <0.0001 | y = 1.72x + 642 | 0.289 | 0.0259 |
| Kappa/lambda | y = −32600x + 18900 | 0.564 | 0.0005 | | 0.0641 | 0.327 |

Correlation of NT-proBNP with the FLC level in 63 patients was determined to be expressed by the following equation:

y=206x-12200, r²=0.481, p<0.0001. NT-proBNP

(4-3) Serum FLC levels determined in a multi-center clinical trial on myocarditis treatment are shown below.
(4-3-1) Table 3 shows the serum FLC levels as measured all the myocarditis patients.

TABLE 3

| | n | kappa (mg/L) | lambda (mg/L) | kappa/lambda |
|---|---|---|---|---|
| Myocarditis | 1,318 | 37.6 ± 19.2** | 72.4 ± 124.2* | 0.64 ± 0.32 |
| Healthy | 17 | 27.9 ± 5.3 | 43.6 ± 8.8 | 0.66 ± 0.16 |
| subjects | | | | |

Mean ± standard deviation,
*p < 0.0005,
**p < 0.0001

(4-3-2) Table 4 shows the effect of infection with hepatitis C virus (HCV) on FLC levels.

TABLE 4

| | n | kappa (mg/L) | lambda (mg/L) | kappa/lambda |
|---|---|---|---|---|
| HCV (+) | 42 | 30.6 ± 14.8* | 173.1 ± 37.1* | 0.27 ± 0.22* |
| HCV (−) | 1,276 | 37.9 ± 19.3* | 69.1 ± 81.7* | 0.66 ± 0.31 |
| Healthy | 17 | 27.9 ± 5.3 | 43.6 ± 8.8 | 0.66 ± 0.16 |
| subjects | | | | |

Mean ± standard deviation,
*p < 0.0001 with respect to healthy subject,
HCV (+, −), p < 0.0001

(4-3-3) Table 5 shows the correlation with NT-proBNP.

TABLE 5

NT-pro-BNP (y)

| | X | r² | p |
|---|---|---|---|
| Kappa | y = 64.8x + 311 | 0.0197 | <0.0001 |
| Lambda | y = 6.58x + 2270 | 0.008496 | 0.0008 |
| Kappa/lambda | y = −2255x + 4202 | 0.0062 | 0.0044 |

(4-3-4) Table 6 shows the FLC levels of cardiomyopathy patients who are positive for hepatitis C virus antibody.

TABLE 6

| | n | kappa | lambda | kappa/lambda |
|---|---|---|---|---|
| Hepatitis C virus | 5 | 42.5 ± 27.2* | 79.9 ± 74.0* | 0.558 ± 0.338 |
| antibody (+) | | | | |
| Healthy subjects | 17 | 27.9 ± 5.3 | 43.6 ± 8.8 | 0.661 ± 0.162 |

*p < 0.05

(4-3-5) Relationship between FLC levels and severity of heart failure

The tested subjects were classified into two categories in terms of severity (light (grades I+II) and grave (grades III+IV)). Each grade denotes a heart function classification on the basis of subjective symptoms graded by the New York Heart Association (NYHA). The results are shown in Table 7.

TABLE 7

| | NYHA heart function classification | | |
|---|---|---|---|
| | Light (grades I + II) | Grave (grades III + IV) | |
| | (n = 17) | (n = 63) | p |
| Kappa | 32.0 ± 15.4 | 40.8 ± 39.2 | NS |
| Lambda | 55.0 ± 44.0 | 93.2 ± 67.9 | 0.03 |
| Kappa/lambda | 0.67 ± 0.31 | 0.46 ± 0.14 | 0.0001 |

### (5) Results

As is clear from Tables 1 to 7, in all the tested patients suffering from heart failure, cardiomyopathy, myocarditis, or a viral disease, the free kappa light chain level and the free lambda light chain level significantly increased, while the kappa/lambda ratio significantly decreased.

### Example 1

### Effect of Administration of an FLC on Encephalomyocarditis Viral Myocarditis (Heart Failure) Model)

### (Procedure)

Encephalomyocarditis virus (1-plaque-forming unit) was injected to the abdominal cavity of each of 4-week-old male DBA/2 mice, and a human free kappa light chain (product of Bethyl laboratories) (10 μg/mouse/day) was subcutaneously injected to the mouse. Instead of the kappa light chain, physiological saline (400 μL) was subcutaneously injected to the mice of a control group. On day 5, the heart was removed from each mouse, and the left ventricle was cut in a horizontal direction, fixed with 10% formalin to prepare paraffin sections. The sections were subjected to hematoxylin and eosin staining and Masson's trichrome staining.

The area of a lesion in cardiac muscle of each mouse was analyzed by means of microanalyzer graphic analysis software (product of Finggal Link Co., Ltd.), whereby the percent lesion area of each cardiac muscle sample was calculated. The obtained data were statistically processed through analysis of variance, and multi-group comparison was performed through the Fischer LSD method.

Survival rate on day 14 was obtained through the Kaplan-Meier viability analysis, and significance was assessed through the Logrank test.

### (Results)

### (1) Area of Cardiac Muscle Lesion:

TABLE 8

| | Cardiac muscle lesion |
|---|---|
| Control group (n = 8) | 39.0 ± 3.8% (mean ± standard error) |
| Human free kappa light chain- | 18.9 ± 2.3% (p < 0.01, vs. control group) |
| administered group (n = 9) | |

As is clear from Table 8, cardiac muscle lesion was significantly mitigated in the human free kappa light chain administration groups.

### (2) Survival Rate

As shown in FIG. 1, all 10 of the tested mice in the control group died within 14 days after injection of encephalomyocarditis virus. In contrast, only two mice died in each human free kappa light chain administration group. Therefore, survival rate of the administration group was significantly improved.

### Example 2

### (Procedure)

The virus level of the heart on day 5 after injection of the virus was determined through the plaque quantitation method employing FL cells (Matsumori A, et al., Circulation. 1985: 71: 834-839).

### (Results)

The virus level of the heart was found to be (1.7±0.5)×10⁶ PFU/g (n=8) in the control group. In contrast, in the human free kappa light chain administration groups, the virus level in the heart was significantly reduced to (0.12±0.05)×10⁶ PFU/g (n=8) (p<0.005).

Thus, a free kappa light chain was found to suppress proliferation of encephalomyocarditis virus in the heart.

### Example 3

### Studies on an Antiviral Effect of an FLC Employing Peripheral Blood Leukocytes Infected with Hepatitis C Virus (HCV)

### (Procedure)

Peripheral blood was collected from patients infected with HCV, from which, mononuclear leucocytes were selectively separated through density gradient centrifugation using Ficoll pack liquid (product of Amersham, U.S.A.). The mononuclear leucocytes were dispersed in an RPMI 1640 medium containing 10% fetal bovine serum to a concentration of 1×10⁶ cells/mL. The thus-produced suspension was added to a 24-well plate at 1 mL/well.

An FLC (1 μg) was added to each well of the plate, and cultured at 37° C. under 5% CO₂ conditions for five days. After completion of culturing, cultured mononuclear leucocytes were collected from each well and applied onto a slide, followed by drying and fixing with ethanol. To the fixed samples, a mouse monoclonal antibody to a core domain protein of HCV (Core A-2 antibody, product of Institute of Immunology Co., Ltd., Takahashi, K., et al., Gen. Virol., 73: 667-672, 1992) (hereinafter referred to as “core antibody”) was added as a primary antibody in an amount of 0.2 mL (20 μg/mL), followed by incubation overnight at 4° C. Subsequently, immunostaining was performed by use of a Bectastein ABC kit for mouse IgG (Burlingame, Calif., U.S.A.) according to the instruction manual of the kit, wherein color was allowed to develop with DAB (diaminobenzidine).

### (Results)

FIG. 2 shows the results. In FIG. 2, color density as obtained from immunostaining of samples of the free kappa light chain-added group (right) was considerably lowered, as compared with the control groups (left).

Thus, the HCV antigen level of the mononuclear leucocytes was found to decrease by the action of a free kappa light chain.

### Example 4

### Studies on Antiviral Effect of FLC Employing HCV-Infected Monocytes

### (Procedure)

Monocytes strain THP-1 (American Type Culture Collection, Manassas, Va., VSA) were dispersed in an RPMI medium containing 10% fetal bovine serum to a concentration of 1×10⁶ cells/mL and cultured in a 24-well plate. To the cultures, serum of a patient infected with HCV (HCV assay: 225 KIU/mL) and a free kappa light chain (1 μg) were added, followed by culturing at 37° C. under 5% CO₂ conditions for five days. Subsequently, cultured THP-1 cells were collected from each well, and immunostaining was performed by use of a core antibody in a manner similar to that of Example 3.

### (Results)

FIG. 3 shows the immunostaining results of THP-1 cells cultured with serum of HCV-infected patient and those of THP-1 cells cultured in a similar medium to which a free kappa light chain has been added. As is clear from FIG. 3, the immunostaining image (right) of THP-1 cells to which a free kappa light chain has been added is a less positive image, as compared with the immunostaining image (left) of THP-1 cells to which no free kappa light chain has been added.

Thus, the HCV antigen level of the monocytes was found to decrease by the action of a free kappa light chain.

1. A method for treating heart disease, comprising administering a therapeutically effective amount of a free immunoglobulin light chain or a constitutive polypeptide thereof to a human being or an animal.

2. The method as described in claim 1, wherein the heart disease is heart failure, cardiomyopathy, myocarditis, or fulminant myocarditis.

3. The method as described in claim 1 or 2, wherein the free immunoglobulin light chain is a kappa chain.

4. A method for treating viral disease, comprising administering a therapeutically effective amount of a free immunoglobulin light chain or a constitutive polypeptide thereof to a human being or an animal.

5. The method as described in claim 4, wherein the free immunoglobulin light chain is a kappa chain.

6. A method for treating heart disease, comprising administering a therapeutically effective amount of a free immunoglobulin light chain or a constitutive polypeptide thereof to a human being or an animal, with the proviso that the immunoglobulin is not derived from an antibody to p53 protein.

7. The method as described in claim 6, wherein the heart disease is heart failure, cardiomyopathy, myocarditis, or fulminant myocarditis.

8. The method as described in claim 6 or 7, wherein the free immunoglobulin light chain is a kappa chain.

9. A method for treating viral disease, comprising administering a therapeutically effective amount of a free immunoglobulin light chain or a constitutive polypeptide thereof to a human being or an animal, with the proviso that the immunoglobulin is not derived from an antibody which catalyzes cleavage of a peptide bond in HIV gp120.

10. The method as described in claim 9, wherein the free immunoglobulin light chain is a kappa chain.

11. The method as described in claim 9 or 10, wherein the viral disease is selected from the group consisting of viral hepatitis, an adenovirus infection, influenza, a herpes infection, viral encephalitis, a cytomegalovirus infection, viral enteritis, and viral pericarditis.

12. The method as described in claim 11, wherein the viral hepatitis is selected from the group consisting of type A, type B, type C, type E, type G, and type TTV.
